(19) United States
(12) Patent Application Publication
(10) Pub. No.: US 2018/0133258 A1
(43) Pub. Date: May 17, 2018

(54) TREATMENT OF CACHEXIA USING STEM CELLS AND PRODUCTS THEREOF

(71) Applicant: Creative Medical Health, LLC, Phoenix, AZ (US)

(72) Inventors: Thomas Ichim, San Diego, CA (US); Amit Patel, Salt Lake City, UT (US)

(73) Assignee: Creative Medical Health, LLC, Phoenix, AZ (US)

(21) Appl. No.: 15/814,284

(22) Filed: Nov. 15, 2017

Related U.S. Application Data
(60) Provisional application No. 62/422,656, filed on Nov. 16, 2016.

(51) Int. Cl. A61K 35/28

(52) U.S. Cl. CPC A61K 35/28

(57) ABSTRACT

The current invention teaches the use of cells possessing regenerative properties, tissues associated with said cells possessing regenerative properties, and products derived from cells possessing regenerative properties for the treatment of cachexia. In one embodiment, cancer or HIV associated cachexia is treated by administration of tissues possessing regenerative cells that have been modified to allow for administration in a manner which is intravenous, subcutaneous or intramuscular while retaining properties associated with cellular regeneration.

### CROSS-REFERENCE TO RELATED APPLICATIONS

The present invention claims priority to U.S. Provisional Application No. 62/422,656, filed Nov. 16, 2016, which is hereby incorporated in its entirety including all tables, figures, and claims.

### FIELD OF THE INVENTION

The invention pertains to the use of immune modulatory cells as a means of reducing cachexia in a patient in need of therapy. More particularly, the invention pertains to the use of mesenchymal stem cells in reducing inflammation associated cachexia. More particularly the invention provides means of augmenting activity of cells to prevent cachexia in a patient in need of therapy. Said patient may be an aging patient, in the case of cachexia of aging, said patient may be a cancer patient, or said patient may be a patient infected with viral or other infections.

### BACKGROUND OF THE INVENTION

Cachexia is a condition associated with muscle wasting particularly presenting in patients with cancer or chronic infections such as HIV. Although studies have shown that inflammatory mediators are causative of cachexia such as TNF-alpha, interventions to inhibit inflammation have been relatively ineffective in clinical situations. One potential reason for this disparity, is the multifactorial nature of cancer associated inflammation.

The process of neoplastic transformation is associated with uncontrolled proliferation of cells, which gives rise to necrosis of de novo tissues, resulting in macrophage infiltration, complement activation, and neoangiogenesis. In situations where this manifestation of neoplasia is occurring at a level where normal physiological processes are disturbed, systemic increases in inflammatory markers are observed, which give rise to exaggerated chronic inflammation, which presents systemic consequences to the overall constitution of the patient.

The majority of cancer patients succumb to disease due to muscle wasting, metabolic abnormalities, and multiple organ failure. Although currently available interventions are focused on reducing tumor burden in a cancer patient, there is a need in the art for interventions which reduce pathologies associated with the impact of tumor masses on the general physiology of the patient. The current invention provides means using mesenchymal stem cells to restore physiological, immunological and metabolic homeostasis in cancer patients, which allows for the possibility of innate and adaptive host responses to initiate, maintain, and execute activities which result in tumor stabilization and in some cases regression.

### DESCRIPTION OF THE INVENTION

The invention provides for administration of regenerative cells, in one embodiment, mesenchymal stem cells, for the inhibition of cachexia. Specifically, the invention provides the use of amniotic fluid stem cells as a therapeutic which upon systemic administration is capable of suppressing tumor derived molecules capable of inducing muscle atrophy and immune inhibition.

The invention teaches that mesenchymal stem cells, for example, amniotic fluid-derived stem cells, are capable of secreting factors which inhibit inflammation, and thereby suppress ability of the host to induce muscle wasting, otherwise known as cachexia. Said cells may be immortal in culture, maintain euploidy for >1 year in culture, share markers with human ES cells, and are capable of differentiating into all three germ layers of the developing embryo, Endoderm, Mesoderm and Ectoderm.

In one embodiment the regenerative amniotic fluid cells are found in the amnion harvested during the second trimester of human pregnancies. It is known that amniotic fluid contains multiple morphologically-distinguishable cell types, the majority of the cells are prone to senescence and are lost from cultures. In one embodiment, fibronectin coated plates and culture conditions described in patent U.S. Pat. No. 7,569,385 are used to grow cells from amniotic fluid harvests from normal 16-18 week pregnancies. The cells of the invention are of fetal origin, and have a normal diploid karyotype. Growth of the amniotic fluid stem cells as described in the invention for use in neurological ischemic conditions results in cells that are multipotent, as several main cell types have been derived from them. As used herein, the term “multipotent” refers to the ability of amniotic fluid regenerative cells to differentiate into several main cell types. The MAFSC cells may also be propagated under specific conditions to become “pluripotent.” The term “pluripotent stem cells” describes stem cells that are capable of differentiating into any type of body cell, when cultured under conditions that give rise to the particular cell type. The Amniotic fluid regenerative cells are preferably isolated from humans. However, the Amniotic fluid regenerative cells may be isolated in a similar manner from other species. Examples of species that may be used to derive the Amniotic fluid regenerative cells include but are not limited to mammals, humans, primates, dogs, cats, goats, elephants, endangered species, cattle, horses, pigs, mice, rabbits, and the like.

The amniotic fluid-derived cells and MAFSC can be recognized by their specific cell surface proteins or by the presence of specific cellular proteins. Typically, specific cell types have specific cell surface proteins. These surface proteins can be used as “markers” to determine or confirm specific cell types. Typically, these surface markers can be visualized using antibody-based technology or other detection methods.

The surface markers of the isolated MAFSC cells derived from independently-harvested amniotic fluid samples were tested for a range of cell surface and other markers, using monoclonal antibodies and FACS analysis. These cells can be characterized by the following cell surface markers: SSEA3, SSEA4, Tra-1-60, Tra-1-81, Tra-2-54, as shown in FIG. 3. The MAFSC cells can be distinguished from mouse ES cells in that the MAFSC cells do not express the cell surface marker SSEA1. Additionally, MAFSC express the stem cell transcription factor Oct-4. The MAFSC cells can be recognized by the presence of at least one, or at least two, or at least three, or at least four, or at least five, or at least six, or all of the following cellular markers SSEA3, SSEA4, Tra-1-60, Tra-1-81, Tra-2-54 and Oct-4.

The MAFSC cultures express very little or no SSEA-1 marker. In addition to the embryo stem cell markers SSEA3, SSEA4, Tra1-60, Tra1-81, Tra2-54, Oct-4 the amniotic fluid regenerative cells also expressed high levels of the cell surface antigens that are normally found on human mesenchymal stem cells, but not normally on human embryo stem cells (M F Pittinger et al., Science 284:143-147, 1999; S Gronthos et al., J. Cell Physiol. 189:54-63, 2001). This set of markers includes CD13 (99.6%) aminopeptidase N, CD44 (99.7%) hyaluronic acid-binding receptor, CD49b (99.8%) collagen/laminin-binding integrin alpha2, and CD105 (97%) endoglin. The presence of both the embryonic stem cell markers and the hMSC markers on the MAFSC cell cultures indicates that amniotic fluid-derived MAFSC cells, grown and propagated as described here, represent a novel class of human stem cells that combined the characteristics of hES cells and of hMSC cells.

In some embodiments of the invention, at least about 90%, 94%, 97%, 99%, or 100% of the cells in the culture express CD13. In additional embodiments, at least about 90%, 94%, 97%, 99%, or 100% of the cells in the culture express CD44. In some embodiments of the invention, a range from at least about 90%, 94%, 97%, 99%, 99.5%, or 100% of the cells in the culture express CD49b. In further embodiments of the invention, a range from at least about 90%, 94%, 97%, 99%, 99.5%, or 100% of the cells in the culture express CD105.

In one particular embodiment of the invention, the amniotic fluid regenerative cells are human stem cells that can be propagated for an indefinite period of time in continuous culture in an undifferentiated state. The term “undifferentiated” refers to cells that have not become specialized cell types. A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors.

The Amniotic fluid regenerative cells may be grown in an undifferentiated state for as long as desired (and optionally stored as described above), and can then be cultured under certain conditions to allow progression to a differentiated state. By “differentiation” is meant the process whereby an unspecialized cell acquires the features of a specialized cell such as a heart, liver, muscle, pancreas or other organ or tissue cell. The Amniotic fluid regenerative cells, when cultured under certain conditions, have the ability to differentiate in a regulated manner into three or more subphenotypes. Once sufficient cellular mass is achieved, cells can be differentiated into endodermal, mesodermal and ectodermal derived tissues in vitro and in vivo. This planned, specialized differentiation from undifferentiated cells towards a specific cell type or tissue type is termed “directed differentiation.” Exemplary cell types that may be prepared from Amniotic fluid regenerative cells using directed differentiation include but are not limited to fat cells, cardiac muscle cells, epithelial cells, liver cells, brain cells, blood cells, neurons, glial cells, pancreatic cells, and the like.

General methods relating to stem cell differentiation techniques that may be useful for differentiating the Amniotic fluid regenerative cells of this invention can be found in general texts such as: Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998); and in Stem cell biology (L. M. Reid, Curr. Opinion Cell Biol. 2:121, 1990), each of which is incorporated by reference herein in its entirety.

Other types of mesenchymal stem cells may be utilized for the practice of the invention, in particular, “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSCcan be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In one embodiment of the invention mesenchymal stem cells are used to treat cachexia associated with aging. In another embodiment cachexia associated factors are used to guide the dose of MSC provided to a patient in which cachexia is aimed to be treated. In another embodiment MSC used are amniotic fluid derived stem cells, said stem cells administered intravenously. In another embodiment amniotic membrane derived stem cells are administered. Amniotic membranes may be used as a source of stem cells by direct administration. In one embodiment amniotic stem cells are administered a concentration of 10-200 million cells intravenously.

Administration of the cachexia inhibiting cells to a human patient can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal or vaginal administration, by perfusion through a regional catheter, or by direct intralesional injection.

In a preferred embodiment, the compositions of the invention are administered by intravenous push or intravenous infusion given over defined period (e.g., 0.5 to 2 hours). The compositions of the invention can be delivered by peristaltic means or in the form of a depot, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered. In particular embodiments, the route of administration is via bolus or continuous infusion over a period of time, once or twice a week.

In other particular embodiments, the route of administration is by subcutaneous injection given in one or more sites (e.g. thigh, waist, buttocks, arm), optionally once or twice weekly. In one embodiment, the compositions, and/or methods of the invention are administered on an outpatient basis. Those skilled in the art will appreciate that dosages can be selected based on a number of factors including the age, sex, species and condition of the subject (e.g., activity of autoimmune disease or disorder), the desired degree of cellular or autoimmune antibody depletion, the disease to be treated and/or the particular antibody or antigen-binding fragment being used and can be determined by one of skill in the art. For example, effective amounts of the compositions of the invention may be extrapolated from dose-response curves derived from in vitro test systems or from animal model (e.g. the cotton rat or monkey) test systems.

Examples of dosing regimens that can be used in the methods of the invention include, but are not limited to, daily, three times weekly (intermittent), weekly, or every 14 days. In certain embodiments, dosing regimens include, but are not limited to, monthly dosing or dosing every 6-8 weeks. Those skilled in the art will appreciate that dosages are generally higher and/or frequency of administration greater for initial treatment as compared with maintenance regimens.

1. A method of treating cachexia comprising the steps of: a) identifying a patient suffering from weight loss; b) administering to said patient a stem cell population, a tissue containing a stem cell population; or c) products produced by a stem cell population or a tissue containing a stem cell population.

2. The method of claim 1, wherein said cachexia is associated with muscle wasting.

3. The method of claim 1, wherein said cachexia is associated with a neoplasia.

4. The method of claim 1, wherein said cachexia is associated with an inflammatory condition.

5. The method of claim 4, wherein said inflammatory condition is a chronic infection.

6. The method of claim 5, wherein said inflammatory conditions is tuberculosis.

7. The method of claim 5, wherein said inflammatory conditions is HIV.

8. The method of claim 1, wherein said patient exhibits an elevation of markers associated with chronic inflammation.

9. The method of claim 8, wherein said markers associated with chronic inflammation include an elevation of C-reactive Protein in peripheral blood as compared to age-matched controls.

10. The method of claim 8, wherein said markers associated with chronic inflammation include an elevation of interleukin-1 in peripheral blood as compared to age-matched controls.

11. The method of claim 8, wherein said markers associated with chronic inflammation include an elevation of TNF-alpha in peripheral blood as compared to age-matched controls.

12. The method of claim 8, wherein said markers associated with chronic inflammation include an elevation of interleukin 6 in peripheral blood as compared to age-matched controls.

13. The method of claim 1, wherein said stem cell population is a mesenchymal stem cell (MSC).

14. The method of claim 13, wherein said MSC is a cell capable of adhering to plastic.

15. The method of claim 1, wherein said MSC is administered in the form of a morselized amniotic membrane tissue.

16. The method of claim 1, wherein said MSC is administered in the form of a single cell suspension.

17. The method of claim 15, wherein said morselized amniotic membrane tissue is treated in a manner to stimulate upregulation of growth factor production, said growth factors stimulatory of anti-cachectic activities.

18. The method of claim 17, wherein said treatment comprises of exposure to hypoxic conditions.

19. The method of claim 17, wherein said treatment comprises of exposure to acidic conditions.

20. The method of claim 17, wherein said treatment comprises of exposure to hypotonic conditions.
